(19) United States
(12) Patent Application Publication
(10) Pub. No.: US 2011/0098737 A1
(43) Pub. Date: Apr. 28, 2011

(54) ANTI-RESTENOSIS DRUG COVERED AND ELUTING BALLOONS FOR VALVULOPLASTY OF AORTIC VALVE STENOSIS FOR THE PREVENTION OF RESTENOSIS

(76) Inventor: Konstantinos Spargias, Smimi Attikis (GR)

(21) Appl. No.: 12/920,278

(22) PCT Filed: Apr. 14, 2008
(86) PCT No.: PCT/GR08/00024

§ 371 Date: Jan. 13, 2011

(30) Foreign Application Priority Data
Apr. 13, 2007 (GR) 20070100224

(51) Int. Cl. A61M 29/00

(52) U.S. Cl. 606/194

(57) ABSTRACT

Described herein are special balloons intended for use in valvuloplasty of stenosed aortic valves, with a potential use for valvuloplasty of other heart valves as well. A feature of these balloons is that the entire or part of their external surface is covered by a drug that is eluted at the valve tissues during the balloon brief contact with them at the time of dilatation. The drug acts against the process of restenosis, which almost uniformly occurs after some time. The shape of the balloon can be the classical cylindrical or an hour-glass shape that facilitates targeted delivery of the drug at the valve tissues.

### BACKGROUND OF THE INVENTION

1. Field of the Invention

The invention generally relates to devices and methods for cardiovascular treatment. More specifically, the invention generally relates to devices and methods for drug delivery to the heart.

2. Description of the Relevant Art

Although aortic valve surgery is the gold standard, life saving treatment for symptomatic aortic stenosis, some patients do not undergo surgery. According to the 2003 Euro Heart Survey (Jung B, et al, Eur Heart J 2003; 24:1231-43) one third of community patients above the age of 75 with symptomatic severe aortic stenosis do not undergo surgery. The most apparent reason for this is the high mortality of the aortic valve replacement (AVR) in this population. The Medicare data in 2003 found a 8.8% in-hospital mortality of AVR in 145,000 patients aged over 65. There are other data showing average mortality of AVR up to 20% in octogenarians.

The incidence of known and severe aortic stenosis in US population in 2006 was 318,000 cases, of which only 74,000 had undergone AVR (based on Nkomo et al, Lancet 2006). According to other US estimates for 2006, of the 161,107 cases of ECHO diagnosed severe aortic stenosis over a 10-year period only 61,658 undergone AVR (based on Loma Linda data). Given the bad prognosis of severe symptomatic aortic stenosis, there has been an unmet need for another supplementary/alternative therapy.

Recently there has been considerable change in the technical approach to balloon aortic valvuloplasty (BAV) owing to the progress of percutaneous aortic valve implantation retrograde from the femoral artery and advances in the interventional hardware. A decrease in the complication rate of BAV over the past 15 years has been reported and has important implications regarding the morbidity associated with percutaneous aortic valve replacement techniques. In a recent series of 104 BAV procedures there were no procedural deaths and the incidence of total vascular complications was 9%. In-hospital, 1-, 2- and 3-year mortality rates were 6%, 44%, 62% and 71%, respectively. In the 1980's NHLBI Balloon Valvuloplasty Registry the procedural mortality was 3% and the in-hospital mortality 11%.

BAV has been well demonstrated to moderately increase aortic valve area by an average of 0.3-0.4 cm² and reduce mean valvular gradient by roughly 50%. This is achieved despite the use of balloon diameters of 18-24 mm, not far smaller from the diameter of the aortic valve ring. Transient valve leaflet and annular stretch undoubtedly contribute to early recoil. The large majority of patients experience immediate symptomatic improvement.

However, the restenosis rates of 42-83% at 5-9 months and consistently >80% at 1 year remain the Achilles heel of the method and is the reason of the dismal long-term event-free and actuarial survival after balloon aortic valvuloplasty that resembles the natural history of untreated aortic stenosis.

It is now recognized that calcific aortic stenosis is a complex cellular process with features of atherosclerosis and biomineralization similar to osteogenesis, which should have specific pathways for targeted inhibition. Similarly, regulated processes may play a role in restenosis following BAV. Although the mechanisms of restenosis are poorly understood, scar formation and heterotopic ossification are believed to play a central role. If specific targeted inhibition of these processes is completely or even partially successful the long-term results of the BAV procedure will improve and it may become an acceptable treatment of aortic valve stenosis in the elderly.

An increasing number of patients are living into the 80s and beyond. Although many of these patients who may have symptomatic stenosis are acceptable candidates for surgical treatment, many are not. Given the uniform desire on behalf of the elderly for improvement in their quality of life, any advance in the BAV procedure that will limit restenosis and improve its long-term results would be welcome in this population.

The first effort to prevent restenosis following BAV was with radiation. The results of the RADAR pilot trial were published in Cathet Cardiovasc Intervent 2006; 68:183-92. This was a series of 20 patients over 80 years of age with an estimated operative mortality risk above 15%. They underwent prophylactic external beam radiation therapy (EBRT) starting the day following the BAV procedure and for 3 days. A total dose of 1200 cGy and 1500-1800 cGy was administered in the low and high dose groups of patients. Restenosis was defined as over 50% late loss of acute gain in aortic valve area. By 1 year the restenosis rate in the low dose group was 30% and in the high dose group 11%, results impressively better than historical controls.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a valvuloplasty balloon/catheter assembly;

FIG. 2 depicts a valvuloplasty balloon/catheter assembly having a drug coating;

FIG. 3 depicts a valvuloplasty balloon/catheter assembly having a partial drug coating;

FIG. 4 depicts a valvuloplasty balloon/catheter assembly having an hour glass shape;

FIG. 5 depicts a valvuloplasty balloon/catheter assembly having a concave middle section;

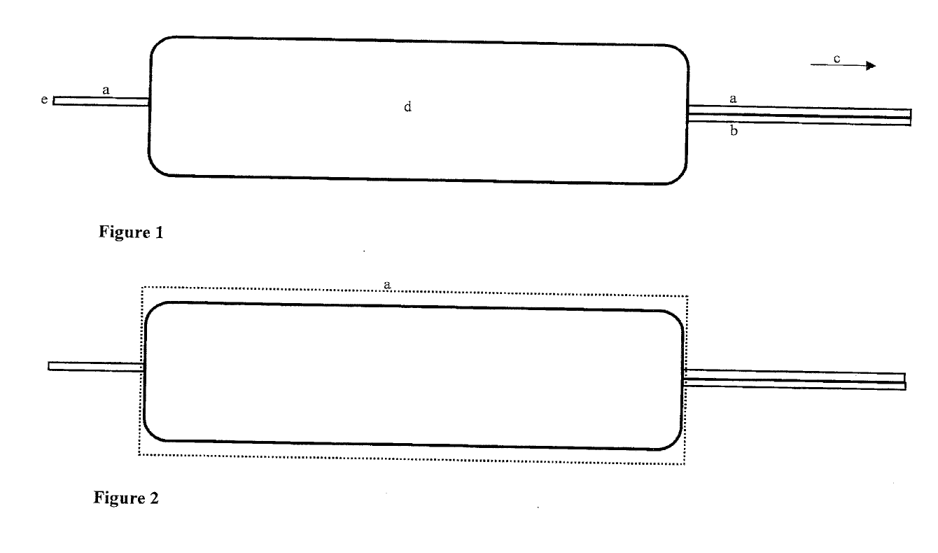

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE EMBODIMENTS

The restenosis in coronary arteries and aortic valve following dilatation of a balloon share certain pathophysiological aspects. My hypothesis is that local antiproliferative drug delivery at the stenotic aortic valve with a balloon is a simple and effective way of preventing restenosis. If the restenosis problem is solved or even attenuated the acceptance/usage of this therapy would be completely revived.

I describe the development of drug-coated balloon for BAV, which elutes the loaded drug to the aortic valve tissues upon contact. There are numerous possibilities to drug coatings for this balloon, from drugs inhibiting scar formation to drugs inhibiting heterotopic ossification.

Since the mechanism of valve restenosis following BAV share certain characteristics with the coronary in-stent restenosis, drugs used for prevention of the latter can be tested for efficacy in the former. Paclitaxel for example is known to inhibit fibroblast migration in vitro and in vivo, and can be the first one to be used for coating a BAV balloon.

Recently, a pilot trial of external beam radiation therapy (EBRT) following BAV in 20 patients demonstrated a sustained significant improvement in the aortic valve area and mean gradient up to 1 year after the procedure (in reality there were only 2 outliers). However, this preventive strategy, even if it is conclusively proved, has certain inherent disadvantages such as the need for liaison with other medical specialties and medical facilities, precision of targeting, and prolonged hospital stay. The effort of restenosis prevention with radiation is reminiscent of the usage of brachytherapy for in-stent restenosis in coronary arteries, before the advent of drug-eluting stents.

In the event that antiproliferative drugs, such as paclitaxel, do not prove to be sufficiently effective, the component of valve restenosis due to heterotopic ossification could be dealt with incorporation in the coating of inhibitors of mineralization/calcification such as MGP, fetuin, osteopontinad and others or oral administration of inhibitors such as phosphate binding drugs (used by all chronic renal failure patients) and NSAA (i.e. the selective cyclooxygenase-2 (COX-2) inhibitor Rofecoxib).

An effective drug-coated balloon for BAV would prevent restenosis with local drug delivery at the aortic valve leaflets at the time of balloon inflation. This method is obviously superior to EBRT, which requires prolonged hospital stay and considerable material and human resources.

If such a balloon proves to prevent restenosis and offers a long-term symptomatic improvement, it will undoubtedly become the treatment of choice in the elderly. The procedure will be much simpler, safer and cheaper compared to the implantation of a percutaneous prosthetic valve. Even partial success with reduction and delay but not elimination of restenosis will be welcome, since repeat BAV can be done in those in need. It is known that repeat BAV is feasible, safe and offers further relief, but cannot be seen as the solution when the restenosis rate is close to 100% after 1 year. However, if the use of the drug-coated balloon attenuates and cuts down the restenosis to acceptable rates, BAV with drug-coated balloon will become readily acceptable.

DIOR (Eurocor, Germany) is a balloon catheter coated with paclitaxel (3 mcg/mm² of balloon surface area) for use in the coronary arteries. It releases 35% of the drug with every 20-second contact with the vessel wall (i.e. 2 such dilatations release almost 70% of the loaded drug). It has been shown to significantly reduce late lumen loss and coronary in-stent restenosis compared with an uncoated balloon.

The aortic valvuloplasty balloon first introduced in the late 70s and today there are plenty plain valvuloplasty balloons manufacturers for use in heart valves. My proposal, which I submit for patenting, is an aortic valvuloplasty balloon that is covered by and elutes an antirestenosis substance (drug) to the aortic valve tissues upon contact with them during its inflation (claim 1).

The drug is released to the aortic valve tissues and exerts its antirestenotic action. The same technology used in the aforementioned balloon catheter (DIOR) or any other drug-covering technology (mechanical or chemical bonding of the drug to the balloon surface) is applied in manufacturing of this balloon.

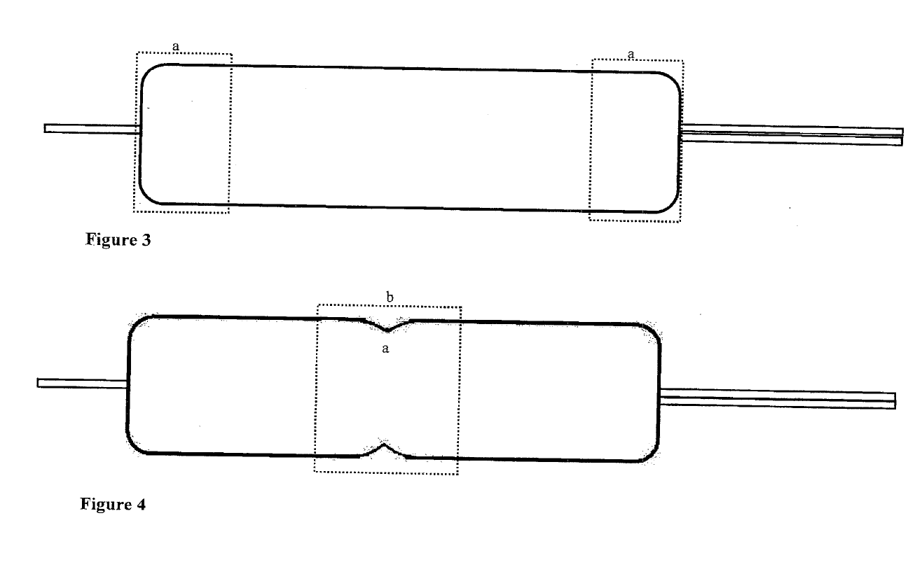

The entire external balloon surface or a part of it will be covered the antirestenotic drug [Claim 2: the entire balloon surface is covered by drug (FIG. 2). Claim 3: the drug covers the middle-part area of the balloon that is coming into direct contact to the aortic valve and the edges remain uncovered (FIG. 3)].

The time of the balloon inflation in BAV cannot exceed 10-15 seconds, but multiple balloon inflations can be applied to reach a total time of balloon-valve contact of almost 1 minute. This allows the almost complete release of the loaded drug to the contacted tissue. If necessary, the quantity of the drug administered at the valve tissues by the balloon may be increased by storing higher dose of the drug at the balloon surface with appropriate technology (i.e. more and/or deeper and/or larger micropores).

A pilot trial in animals is designed to prove the concept of local drug delivery at the aortic leaflets followed by studies with experimental models of animal aortic valve stenosis and ultimately studies in humans with aortic valve stenosis.

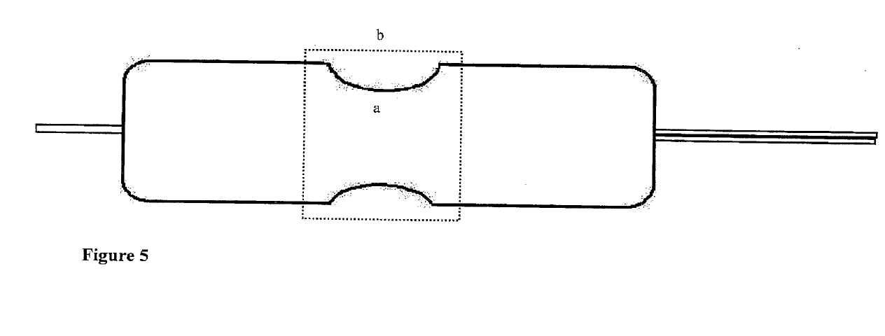

This balloon will achieve contact and drug release at the inferior/external surfaces of the aortic valve, which have considerably larger area compared to the superior/internal surface for geometrical reasons. The safety of drug delivery at other endothelial sites in the vicinity (i.e. aortic root and left ventricular outflow tract-LVOT) will be assessed. If animal or initial human experience raises concerns about possible side effects of paclitaxel or other drugs apposition at the aortic root/LVOT endothelium, a hour-glass shaped balloon is developed with the drug loaded only in the middle slimmer part (waist) of the balloon (claim 4). With such a design the drug will be administered in a targeted manner at the aortic valve tissue only, and contact with other structures will be avoided. In addition, such a shape will act protectively with regards to any premature release of the drug into the blood flow. Claim 5 describes a balloon that when inflated forms a circular crease at its middle part, the surface of which is covered by the drug (FIG. 4). In BAV the pathological aortic valve contacts this balloon at the level of its drug-covered crease. Claim 6 describes a balloon similar to that of claim 5, the only difference being that instead of forming a crease it forms a circular concave perimeter (FIG. 5).

All previously described balloons can be used for valvuloplasty of other heart valves (claim 6).

Phosphate binding drugs could be used for inhibition of the valve/vascular calcification component of restenosis in these studies.

Other antirestenotic and anticalcification drugs (alone or in combination) can be tested for local delivery with a coated balloon.

What is claimed is:

1. A balloon for use in valvuloplasty of aortic valve stenosis comprising a valvuloplasty balloon/catheter assembly comprising two lumens running through the entire shaft of the assembly and reaching to the assembly's external end that remains outside of the patient's body:
wherein one lumen is used for inflation of a balloon that is mounted to the opposite end of the assembly, and wherein the other lumen is used for passing a guide-wire over which the balloon is advanced from an entry site to an application site, wherein a drug against restenosis, coats the external surface of this balloon, and wherein the antirestenotic drug is delivered from the external surface of the balloon to the aortic valve tissues during the inflation of the balloon in performing valvuloplasty.

2. The balloon of claim 1, wherein the entire external balloon surface is coated with an antirestenotic drug.

3. The balloon of claim 1, wherein the external balloon surface is partially coated with an antirestenotic drug at the level of its middle segment that contacts the aortic valve tissues upon inflation, and wherein the balloon surfaces at both ends of the assembly remain uncoated.

4. The balloon of claim 1, wherein the balloon takes the shape of an hour-glass shape when inflated such that a middle segment has a smaller diameter than the diameter of the ends, and wherein only the surfaces of the middle segment are coated with an antirestenotic drug.

5. The balloon of claim 4, wherein the middle segment has a crease shape.

6. The balloon of claim 4, wherein the middle segment has a concave shape.

7. The balloon of claim 1, wherein the balloon is configured to be used for valvuloplasty of non-aortic heart valves.
